(19) United States
(12) Patent Application Publication
(10) Pub. No.: US 2025/0101407 A1
(43) Pub. Date: Mar. 27, 2025

(54) COMPOSITIONS AND METHODS FOR ISOLATION AND ENRICHMENT OF CIRCULAR RNAS AND KITS THEREOF

(71) Applicant: PRASHANT KARBHARI KHADE, CARLSBAD, CA (US)

(72) Inventor: PRASHANT KARBHARI KHADE, CARLSBAD, CA (US)

(21) Appl. No.: 18/893,105

(22) Filed: Sep. 23, 2024

Related U.S. Application Data
(60) Provisional application No. 63/540,088, filed on Sep. 24, 2023.

(51) Int. Cl. C12N 15/10; C12N 9/12; C12N 9/14

(52) U.S. Cl. CPC C12N 15/1003; C12N 9/1205; C12N 9/1241; C12N 9/14

(57) ABSTRACT

Present invention provides components, compositions, kits, and methods for enrichment and purification of circular RNAs (circRNAs) from circRNAs containing RNA compositions. Specifically, the invention of the present invention concerns the use of 3′ and 5′ free ends of linear RNA molecules for 3′- and 5′-adaptors addition. 3′- and 5′-adaptors comprise of affinity groups, which, when attached to the free ends of linear RNA molecules, facilitate their efficient depletion. This invention is applicable to RNA compositions containing circRNAs, and can be used for the enrichment and purification of specific or complete circRNA repertoires from all cell types and organisms. Our invention enhances the sensitivity and specificity for detecting a complete and unbiased repertoire of circRNAs present in the sample.

### CROSS-REFERENCE TO RELATED APPLICATIONS

This non-provisional application claims benefit of provisional application U.S. Ser. No. 63/540,088 filed Sep. 24, 2023, the entirety of which is hereby incorporated by reference.

### FEDERAL FUNDING LEGEND

This invention was made with government support under the Small Business Innovation Research (SBIR) grant 1R43HG013073-01A1 awarded by the National Institute of Health (NIH). The government has certain rights in the invention.

### INCORPORATION OF SEQUENCE LISTING

A sequence listing is provided herewith containing the file named “D2234NP_SEQ_092324.xml” created on Sep. 23, 2024 and having a size of 37 kilobytes. The contents of the Sequence Listing XML are incorporated by reference herein in its entirety.

### FIELD OF THE INVENTION

The present invention relates generally to the field of molecular biology. More specifically, it relates to the field of circular RNAs, with a particular focus on an unbiased, rapid, and efficient method for enriching the complete repertoire of circular RNAs from both naturally occurring and artificially created RNA-containing compositions of any cell or organism. The methods, compositions, and kits are applicable for a variety of purposes, including research, diagnostics in humans or non-humans, as well as therapeutic applications.

### BACKGROUND OF THE INVENTION

CircRNAs are closed loop single stranded RNA transcripts generated by back splicing from a single pre-mRNA called intronic, exonic and intron-exon circRNAs. Closed loop structure renders them more stable than their linear RNA counterpart. Thousands of circRNAs have been reported since their discovery >40 years ago from human transcriptome. [1-5] Emerging evidence indicated circRNAs are broadly expressed in eukaryotic cells, show cell type- and tissues-specificity; and their expressions are often conserved across species. [6] circRNAs have been implicated in hepatocellular carcinoma [7], neurological diseases [8], innate immunity [9,10], and lung fibrosis [11] demonstrating their role in cellular physiological and pathological pathways. Further, viruses have been shown to encode circRNAs providing a strong impetus to understand their sequences, and roles in viral infections; and in antiviral immune responses. [12,13]

CircRNAs attracted great potential as a disease diagnostic marker due to their high stability in body tissues and fluids, and they are expressed in a tissue- and development-specific manner. [14] Initially discovery of circRNAs proposed their role as a decoy for microRNA which contain plenty of miRNA binding sites leading to decreasing of the functional miRNA molecules. Further, they are involved in regulating of gene transcription, mRNA splicing, binding to RNA-binding proteins and translating into proteins. In the end their role is very well accepted in regulating gene expression. [6]

There is a continued strong interest in identifying and analyzing the expression of circular RNA molecules to gain insights into cellular responses to environmental factors, differentiation, and other biological processes in both normal and abnormal eukaryotic cells. A major focus is on studying disease-related circular RNA molecules in eukaryotic cells to gain insights into various diseases, with the ultimate goal of developing treatments or preventive measures to stop or slow disease progression.

Some of the circRNAs are expressed higher level than their associated linear RNA but majority of them are expressed at very low levels. [1,2] Many circRNAs are identified using RNA-seq followed by computational platform to find backsplicing junctions. However, due to low abundance of many circRNAs; detection by RNA-seq methods and difficulty in their annotation of start-and-end coordinates by computation methods are preventing identification of complete circRNA repertoire. [15] Further, majority of circRNAs share similar body sequences with their linear RNA counterpart, hence it is always difficult to find complete sequence of circRNA by RNA-seq. Additionally, very little overlap was observed in the circular RNAs predicted by various computational pipelines.

Currently, ribonuclease R (RNase-R) is a widely used method to enrich circRNAs from total RNA. RNase-R is an exoribonuclease that can hydrolyze linear RNA from 3′→5′ direction. However, several abundant cellular RNAs such as tRNA and small nuclear RNA are poor substrate for RNase-R degradation due to their highly structured ends.

Existing methods, such as RNase-R digestion and rRNA depletion, are ineffective at fully eliminating linear RNAs, thereby hindering the detection and analysis of the less abundant circRNA landscape. [16, 17] Linear RNA degradation using RNase-R, combined with rRNA or polyA depletion, has been utilized to enrich circRNAs. However, the resulting enriched samples display minimal overlap in the circRNAs identified. Additionally, many highly structured linear RNAs are not fully digested by RNase-R, leading to their presence in the enriched circRNA fraction and complicating the comprehensive identification of the circRNA repertoire. To address these challenges, several modifications to the RNase-R digestion process have been developed. One such approach involves adding a poly(A) tail to the 3′ end of RNA, followed by the depletion of linear RNAs using oligo dT biotinylated beads and RNase-R digestion to enrich circRNAs. However, many studies have highlighted the sensitivity of certain circRNAs to RNase-R digestion. [5, 18-21] Although, RNase-R treatment effectively enriches a broad spectrum of circRNAs, however, RNase-R-sensitive circRNAs are degraded during the process, reducing the overall efficiency of this method.

Additionally, circRNAc unlike miRNAs, tRNAs and small RNAs are not easily separated by gel or size electrophoresis. Hence, we need to use alternative biochemical methods for their enrichment and isolation.

As a result, ongoing efforts are focused on developing an unbiased and more effective circRNA enrichment platform that can accurately capture the full repertoire of circRNAs, along with their sequences and abundances. However, the lack of easy-to-use kits for circRNA isolation has greatly impeded progress, slowing the discovery of circRNAs and limiting the understanding of their roles in many poorly understood diseases. Identification of complete unbiased repertoire will provide deeper insights into splicing mechanisms and significantly enhance our understanding of the complex and dynamic nature of the transcriptome.

Therefore, identification of circRNA landscape offer a vast opportunity to explore and model complex cellular processes. Given the power and potential for widespread use, there is an urgent and unmet need to develop a platform, that is less time consuming, free of artefacts, and inexpensive. Furthermore, identification of novel circRNA will adds to the growing repertoire of regulatory functions in gene expression and present an opportunity to elucidate different physiological and pathological conditions.

Therefore, the objective of the present invention is to provide methods and compositions that enable the enrichment of the complete circRNA repertoire by eliminating the RNase-R based enrichment process.

Prior to the present invention, no methods were known in the art for selectively modifying the free 3′ and 5′ ends of linear RNAs to enable their depletion in circular RNA enrichment or isolation.

### SUMMARY OF THE INVENTION

The object of the present invention is to provide methods, compositions, and kit thereof which concerns a quick and easy enrichment of circRNAs. CircRNAs are single-stranded RNA molecules that form a closed continuous loop by joining their 3′ and 5′ ends, unlike linear RNAs, which have free 3′ and 5′ ends. Circular RNAs due to their closed loop structures; their 5′ and 3′ ends are not available for ligation or addition reaction.

Present invention is directed to a method of enriching circular ribonucleic acid (circRNA) molecules in a total RNA sample. The method comprising the steps of: (a) obtaining the total RNA sample comprising the circRNA and non-circRNA molecules; (b) contacting the sample with one or more adaptors comprising an affinity molecule under conditions to produce non-circRNA molecules ligated with the affinity molecule at one or more of 3′-end and 5′-end of the non-circRNA molecules; (c) binding the ligated non-circRNA molecules to a binding matrix comprising the affinity molecule binding substance to capture and immobilize the ligated non-circRNA molecules from the total RNA sample; (d) depleting the immobilized non-circRNA molecules by removing the binding matrix from the sample and thereby enriching the circRNA molecules in said total RNA sample.

Present invention is further directed to a method of enriching circular ribonucleic acid (circRNA) molecules in a total RNA sample, the method comprising the steps of: (a) obtaining the total RNA sample comprising the circRNA and non-circRNA molecules; (b) contacting the sample with one or more adaptors comprising a biotin label under conditions to produce non-circRNA molecules ligated with the biotin label at one or more of 3′-end and 5′-end of the non-circRNA molecules; (c) binding the ligated non-circRNA molecules to streptavidin-coated magnetic beads to capture and immobilize the ligated non-circular RNA molecules via the biotin label; (d) depleting the immobilized non-circRNA molecules by separating the magnetic beads from the sample and thereby enriching the circRNA molecules in said total RNA sample.

Present invention is directed further still to a method of enriching circular ribonucleic acid (circRNA) molecules in a total RNA sample, the method comprising the steps of: (a) obtaining the total RNA sample comprising the circRNA and non-circRNA molecules; (b) contacting the sample with a reagent mixture such that one or more functionally reactive groups are generated at one or more of 3′-end and 5′-end of the non-circRNA molecules, wherein said one or more functionally reactive groups are selected from the group consisting of 3′-hydroxyl, 5′-hydroxyl, 3′-phosphate, and 5′-phosphate; (c) contacting the sample with one or more adaptors comprising a biotin label under conditions to produce non-circRNA molecules ligated with the biotin label at one more of 3′-end and 5′-end of the non-circRNA molecules; (d) binding the ligated non-circRNA molecules to streptavidin-coated magnetic beads to capture and immobilize the ligated non-circular RNA molecules via the biotin label; (e) depleting the immobilized non-circRNA molecules by separating the magnetic beads from the sample and thereby enriching the circRNA in said total RNA sample.

The present invention provides methods for adding or linking a 3′ adaptor to the 3′-end of linear RNAs to facilitate their depletion in circular RNA containing sample. The present invention also provides methods for adding or linking a 5′ adaptor to the 5′-end of linear RNAs to enable their depletion in circular RNA containing sample.

In one embodiment, the method involves adding a 3′ adaptor to the 3′-hydroxyl end of linear RNA molecules in a sample. The method includes incubating the sample with a molar excess of a 3′ adaptor containing a 5′-APP (5′-adenylated modification) end, along with T4 RNA ligase-2 truncated, under specific conditions and for a sufficient duration to allow the 3′ adaptor to be efficiently added or linked to the free 3′-hydroxyl end of the linear RNA molecules.

In one embodiment, adding 5′ adaptor to the 5′ monophosphate end of linear RNA molecules in a sample. The method includes incubating the sample with the molar excess of an 5′ adaptor having 3′-hydroxyl end, along with T4 RNA ligase-1 under specific conditions and for a sufficient duration to allow 5′ adaptor is added to the free 5′-monophosphate end of linear RNA molecules.

In the foregoing embodiments, the 3′ and 5′ adaptors comprise an affinity molecule, and the method employs a solid support with an affinity-binding substance capable of binding to said molecules. This enables the linear RNA molecules, when linked to the 3′ and 5′ adaptors containing affinity molecules, to be effectively captured on the solid support.

In a further embodiment, the linear RNA molecules present in the sample may contain a 2′-3′ cyclic phosphate group. However, a 3′ adaptor is only added to the 3′ end when a free 3′-hydroxyl group is available. The method includes the additional step of treating the total RNA sample with T4 polynucleotide kinase (T4 PNK) under conditions and for a duration sufficient to convert RNA molecules with a 2′-3′ cyclic phosphate group into RNA molecules with a free 3′-hydroxyl end, while also generating a 5′-monophosphate end. Further, T4 PNK converts the 3′-monophosphate into a free 3′-hydroxyl end to facilitate addition of 3′-adaptor. Additionally, T4 PNK converts 5′-hydroxyl into 5′-monophosphate in the presence of ATP to facilitate addition of 5′-adaptor to 5′-monophosphate end. T4 PNK treatment can be carried out either separately or within the same reaction to facilitate the efficient addition of 3′ and 5′ adaptors.

In some embodiment, the RNA sample prior to 3′ and 5′ adaptors addition may comprise step (a) enzymatically treating 5′-triphosphate and diphosphate of linear RNA to produce the 5′-monophosphate RNA molecule; (b) enzymatically treating 5′-hydroxyl of linear RNA molecules with T4-PNK to produce 5′-monophosphate RNA molecule; and (c) Purifying generated 5′-monophosphate RNA for 5′ adaptor addition.

In some embodiment, the RNA sample prior to 3′ adaptor addition a method may comprise step (a) enzymatically adding a poly(A) tail to the 3′ end of linear RNA molecules ensures that any structural perturbation or modification at the 3′ end does not interfere with the efficiency of 3′ adaptor addition by contacting the RNA sample with poly(A) polymerase and ATP for sufficient time; (b) Purifying generated poly(A) RNA for 3′ adaptor addition.

In one embodiment, total RNA isolated from Hela cell. During or after total RNA isolation it is treated with DNase-I (RNase-free) DNase-I is enzyme known to degrade single and double stranded DNA from the sample. This step plays a crucial role in reducing DNA contamination, which can interfere with the accuracy of downstream applications like RT-qPCR and RNA sequencing (RNA-seq). By removing or minimizing the presence of DNA, the process helps ensure that only RNA is amplified and sequenced, leading to more reliable and specific data. In RT-qPCR, DNA contamination can result in false-positive signals, as both RNA and residual DNA may be amplified, skewing quantification results. Similarly, in RNA-seq, DNA contamination can generate misleading reads, falsely appearing as expressed genes or transcripts, thereby affecting the quality and interpretation of the sequencing data. Effectively reducing DNA contamination increases the specificity and precision of the developed methods, thereby enhancing overall data integrity.

In one embodiment, 5′ and 3′-ends of total RNA are prepared for two-way ligation reaction by treating with T4 Polynucleotide Kinase (T4 PNK) in presence or absence of ATP. T4 PNK remove the 3′-monophosphate of RNA if present to turn it to 3′-hydroxyl and add 5′-monophosphate stoichiometrically in the absence of ATP. Modification of the 3′ and 5′ ends ensures that majority of linear RNA molecules possess free 3′-OH and 5′-monophosphate groups, making them suitable for 3′ and 5′ ligation or addition reactions. T4 PNK treatment can be performed during or before 3′ and or and 5′ adaptor ligation or addition. Here, different end modification methods are feasible for 5′ and 3′ linear RNA ends.

In some embodiments, biotinylated 3′- and 5′-adaptors are ligated to linear RNA molecules, which are then tethered to streptavidin-coated magnetic beads. The biotinylated linear RNA molecules are captured and immobilized on streptavidin-coated magnetic beads, allowing for the enrichment of circular RNA molecules in the supernatant. Optionally, after the enrichment process, the circular RNAs present in the supernatant are further purified using standard or specialized RNA isolation kits.

The present disclosure is a method of enriching circular RNAs from total RNA or rRNA depleted RNA or circRNA containing RNA composition. In non-limiting examples of present invention, the total RNA or sample RNA is isolated from the group consisting of eukaryotic cells, blood, plasma, serum, saliva, cerebrospinal fluid, urine, stool, blood and a combination thereof.

In some embodiments the RNA from the host is circulating cell free circular RNAs is isolated using present invention. In another aspect, the present disclosure is a method for isolating circular RNA sample from host, in which (a) isolating RNA sample from the host or any other source; (b) a step of preparing RNA sample for efficient ligation; (c) enriching circular RNA using disclosed invention methods or combination.

The present disclosure provides several methods of circular RNA enrichment. Such techniques can be used alone or in combinations with other known or invented methods.

The compositions, methods and kits of the invention have wide applicability. For example, enriched circular RNA can be used to synthesize its full-length cDNA for the purpose of long read sequencing or various types of RNA sequencing is employed to identify the full-length sequences, nucleotide modifications, structure, and function.

In some embodiments may comprise sequencing of enriched RNA molecules. The sequencing method may be implemented in a variety of different ways so that a particular population of enriched RNA molecules is sequenced.

### BRIEF DESCRIPTION OF THE DRAWINGS

Those skilled in the art will recognize that the following drawings are provided solely for illustrative purposes. These drawings are not meant to restrict or define the scope of the present teachings in any way.

FIG. 1 provides a schematic representation of circular RNA enrichment from a total RNA sample. The enrichment method is detailed in Example 1.

FIG. 2 demonstrates depletion efficiency of various linear RNA transcripts. The method demonstrated in this invention enables highly efficient depletion of linear RNAs. Data normalized to control and mean±SD n=3.

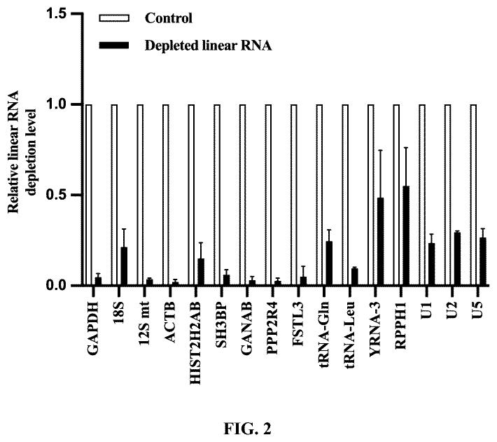

FIG. 3 demonstrate enrichment of various circular RNAs. Circular RNAs enrichment was carried as demonstrated in example 1. The method as disclosed in the present invention provides the highly efficient enrichment of circular RNAs. Data normalized to control and mean±SD n=3.

FIG. 4 demonstrates the classification of analyzed sequence reads from RNA-seq libraries, categorizing the sequencing reads into various RNA types, such as protein coding RNA, mitochondrial RNA (mt-RNA), long non-coding RNA (lncRNA), and other types of RNA. The compositions and methods disclosed in the present invention (Example 1) exhibited significantly higher percentages of circular RNA reads compared to control total RNA.

FIG. 5 demonstrates the number of unique circular RNAs enriched using various enrichment methods described in this invention. As demonstrated in this invention, the sequential use of both 3′ and 5′ adaptors are more effective in depleting linear RNAs compared to the use of either 5′ or 3′ adaptors individually.

FIGS. 6A-6C illustrates an embodiment for preparing the 3′ and 5′ ends of linear RNA to facilitate efficient adaptor ligation, thereby promoting their depletion. Generating linear RNA 5′-monophosphate ends by RNA pyrophosphohydrolase (RppH) enzymes by treating total RNA sample is shown in FIG. 6A (Example 5). Generating linear RNA 3′ poly(A) ends from total RNA sample by poly(A) polymerase treatment is shown in FIG. 6B (Example 6). Generating linear RNA 5′-monophosphate and 3′-hydroxyl ends by treating total RNA sample with T4 PNK is shown in FIG. 6C (Example 7).

FIG. 7 demonstrates the efficiency of linear RNA depletion by modifying their 3′ and 5′ ends with enzymes to enhance ligation, thereby improving depletion. The variation in 3′ and 5′ ends affects the enrichment of different linear RNAs, showing particularly high efficiency for highly abundant linear RNA.

FIG. 8 demonstrates the enrichment of the circUGP2 RNA through the modification of the 3′ and 5′ ends of linear RNA molecules using enzymes, enhancing circular RNA enrichment. The results indicate that the compositions and methods employed in the invention effectively enrich circUGP2 RNA.

FIG. 9 illustrates the enrichment of circASXL1 RNA through the modification of the 3′ and 5′ ends of linear RNA molecules using enzymes, enhancing circular RNA enrichment. The results demonstrate that the compositions and methods utilized in the invention effectively enrich circASXL1 RNA.

### DETAILED DESCRIPTION OF THE INVENTION

### Definitions

To facilitate an understanding of the present invention, a number of terms and phrases are defined herein:

The term circular RNA (circRNA) refers to a form of single-stranded RNA that, in contrast to linear RNA, forms a continuous covalently closed loop. The circular structure is created when the 3′ and 5′ ends of the RNA molecule are joined together, forming a continuous, stable circle. The terms circular RNA and circRNA have been used interchangeably in the present invention, and both terms are intended to have the same meaning.

The term non-circular RNA (non-circRNA) composition refers to a collection of RNA molecules that exist in a linear form, possessing distinct 5′ and/or 3′ ends, as opposed to a continuous covalently closed-loop or circular structure.

The term “ligase” refers to a type of enzyme that catalyzes the joining (ligation) of two molecules, typically with the use of energy derived from ATP or another nucleotide. Ligases are crucial in various biological processes where the formation of covalent bonds between molecules is required.

The term “kinase” refers to a type of enzyme that catalyzes the transfer of a phosphate group from a high-energy molecule like ATP to a specific substrate, typically proteins, lipids, or nucleotides. This process, known as phosphorylation.

The term “enzyme” refers to a biological catalyst, typically a protein, that accelerates chemical reactions in living organisms without being consumed in the process.

The term “T4 polynucleotide kinase (T4 PNK)” refers to an enzyme that transfer a phosphate group from ATP to the 5′-hydroxyl end of a nucleic acid (DNA or RNA). This enzyme also has a 3′-phosphatase activity, allowing it to remove phosphate groups from the 3′ end of nucleic acids.

The term “T4 RNA ligase 2 truncated” refers to an enzyme and its preference for ligating a pre-adenylated (pre-activated) donor to the 3′-hydroxyl group of a single-stranded RNA acceptor, without the need for ATP.

The term “T4 RNA ligase 1” refers to an enzyme that catalyze the formation of a phosphodiester bond between the 3′-hydroxyl (3′-OH) group of one RNA molecule and the 5′-monophosphate (5′-P) group of another RNA molecule in the presence of ATP.

The term “RNase R” refers to an exonuclease enzyme that specifically degrades linear RNA by digesting it from the 3′ to 5′ end, while leaving circular RNA (circRNA) and other structured RNAs largely intact.

The term “linear RNA molecules” refers to RNA strands with distinct 5′ and 3′ ends, lacking the circular closed end structure found in circular RNA. These molecules are characterized by their open-ended, non-looped configuration and can include messenger RNA (mRNA), long non-coding RNA (lncRNA), and other RNA species that exist in a linear form.

The term “adaptor” refers to a short, synthetic piece of single stranded DNA or RNA that is attached to the ends of DNA or RNA fragments during the next generation sequencing library preparation process.

The term “3′ adaptor” refers to a short synthetic sequence of nucleotides that is specifically ligated to the 3′ end of RNA or DNA molecules during library preparation for sequencing or other molecular biology techniques.

The term “5′ adaptor” refers to a short synthetic sequence of nucleotides that is specifically ligated to the 5′ end of RNA or DNA molecules during library preparation for sequencing or other molecular biology techniques.

The term “oligonucleotide” refers to a short sequence of nucleotides, typically composed of between 2 to 100 nucleotides. These molecules can be composed of DNA, RNA, or synthetic analogs and are widely used in molecular biology, genetics, and biochemistry for various applications.

The terms “adaptor” and “oligonucleotide” have been used interchangeably in the present invention, and both terms are intended to have the same meaning.

The term “Poly(A) polymerase (PAP)” refers to an enzyme that adds a polyadenylate poly(A) tail to the 3′ end of an RNA molecule. The poly(A) tail is a sequence of adenosine monophosphate (AMP) residues.

The term “affinity molecule” refers to a molecule that specifically binds to a target molecule with high selectivity and strength through non-covalent interactions, such as hydrogen bonds, ionic bonds, van der Waals forces, and hydrophobic interactions.

The term “Biotin” refers to a affinity molecule that has an exceptionally strong and specific binding typically to streptavidin or avidin.

The term “biotinylates oligo or adaptor” refers to an oligonucleotide that has been biotinylated meaning a biotin molecule has been covalently attached to the oligonucleotide. This modification allows the oligonucleotide to be captured, detected, or purified through the strong binding interaction between biotin and an affinity molecule, typically streptavidin or avidin.

The term “Streptavidin magnetic beads” refers to a magnetic particle coated with streptavidin, a protein that has an extremely high affinity for biotin. These beads are widely used in molecular biology, biotechnology, and diagnostic applications for affinity purification.

In one embodiment, present invention discloses a method of enriching circular ribonucleic acid (circRNA) molecules in a total RNA sample. The method comprising the steps of: (a) obtaining the total RNA sample comprising the circRNA and non-circRNA molecules; (b) contacting the sample with one or more adaptors comprising an affinity molecule under conditions to produce non-circRNA molecules ligated with the affinity molecule at one or more of 3′-end and 5′-end of the non-circRNA molecules; (c) binding the ligated non-circRNA molecules to a binding matrix comprising the affinity molecule binding substance to capture and immobilize the ligated non-circRNA molecules from the total RNA sample; (d) depleting the immobilized non-circRNA molecules by removing the binding matrix from the sample and thereby enriching the circRNA molecules in said total RNA sample.

In an aspect of the invention, the method of enriching circRNA molecules in the total RNA sample includes treating the sample with a reagent mixture such that functionally reactive groups are generated at one or more of 3′-end and 5′-end of the non-circRNA molecules to ligate the adaptors containing the affinity molecule. Functionally reactive groups of the non-circRNA molecules is selected from the group consisting of 3′-hydroxyl, 5′-hydroxyl, 3′-phosphate, and 5′-phosphate.

In a non-limiting example, the affinity molecule is biotin and the binding matrix comprising the affinity molecule binding substance is streptavidin magnetic beads.

In one embodiment, present invention discloses a method of enriching circular ribonucleic acid (circRNA) molecules in a total RNA sample, the method comprising the steps of: (a) obtaining the total RNA sample comprising the circRNA and non-circRNA molecules; (b) contacting the sample with one or more adaptors comprising a biotin label under conditions to produce non-circRNA molecules ligated with the biotin label at one or more of 3′-end and 5′-end of the non-circRNA molecules; (c) binding the ligated non-circRNA molecules to streptavidin-coated magnetic beads to capture and immobilize the ligated non-circular RNA molecules via the biotin label; (d) depleting the immobilized non-circRNA molecules by separating the magnetic beads from the sample and thereby enriching the circRNA molecules in said total RNA sample.

In an aspect of the invention, the method of enriching circRNA molecules in the total RNA sample includes treating the sample with a reagent mixture such that functionally reactive groups are generated at one or more of 3′-end and 5′-end of the non-circRNA molecules to ligate the adaptors containing the affinity molecule. Functionally reactive groups of the non-circRNA molecules is selected from the group consisting of 3′-hydroxyl, 5′-hydroxyl, 3′-phosphate, and 5′-phosphate.

In one embodiment, present invention discloses a method of enriching circular ribonucleic acid (circRNA) molecules in a total RNA sample, the method comprising the steps of: (a) obtaining the total RNA sample comprising the circRNA and non-circRNA molecules; (b) contacting the sample with a reagent mixture such that one or more functionally reactive groups are generated at one or more of 3′-end and 5′-end of the non-circRNA molecules, wherein said one or more functionally reactive groups are selected from the group consisting of 3′-hydroxyl, 5′-hydroxyl, 3′-phosphate, and 5′-phosphate; (c) contacting the sample with one or more adaptors comprising a biotin label under conditions to produce non-circRNA molecules ligated with the biotin label at one more of 3′-end and 5′-end of the non-circRNA molecules; (d) binding the ligated non-circRNA molecules to streptavidin-coated magnetic beads to capture and immobilize the ligated non-circular RNA molecules via the biotin label; (e) depleting the immobilized non-circRNA molecules by separating the magnetic beads from the sample and thereby enriching the circRNA in said total RNA sample.

In an aspect of the invention, the method of enriching circRNA molecules in the total RNA sample includes treating the sample with a reagent mixture such that functionally reactive groups are generated at one or more of 3′-end and 5′-end of the non-circRNA molecules to ligate the adaptors containing the affinity molecule. Functionally reactive groups of the non-circRNA molecules is selected from the group consisting of 3′-hydroxyl, 5′-hydroxyl, 3′-phosphate, and 5′-phosphate.

### General Embodiments of the Invention

The invention related to novel methods, compositions, and kits for selectively enriching population of circular RNA molecules in a mixture or composition of RNA. The RNA mixture or composition may contain a combination of linear RNA molecules with open 3′ and 5′ ends, as well as circular RNA molecules with closed 3′ and 5′ ends.

Total RNA from eukaryotic cells comprises various linear RNA molecules, some of which possess specific chemical groups at their 3′ or 5′ ends. For instance, eukaryotic non-coding RNAs (ncRNAs) commonly have a 5′ triphosphate group. Similarly, bacterial mRNAs also contain a 5′ triphosphate group. Further, small ribosomal RNAs, such as 5S and 5.8S rRNAs, as well as transfer RNAs (tRNAs) from both prokaryotes and eukaryotes, typically feature a 5′ triphosphate group. Additionally, fragmented RNAs may have random chemical moieties, such as hydroxyl or monophosphate groups at their 3′ or 5′ ends.

Each class of linear RNA have a particular group or chemical moiety at their 3′ and 5′-ends. Chemical moieties at 5′ end includes 5′-monophosphate, 5′-diphosphate, 5′-monophosphate, 5′-Capped RNA etc. Chemical moieties at 3′ end includes 3′-monophosphate, 2′-3′ cyclic phosphate, 3′-hydroxyl etc. A common characteristic of most linear RNAs is the presence of a 3′ hydroxyl end. Many of the chemical moieties or functional groups present at the 3′ and 5′ ends of linear RNAs may be employed to facilitate efficient depletion.

Circular RNA forms a closed-loop structure, and therefore lacks free 3′ and 5′ ends. The accessibility of the 3′ and 5′ ends of linear RNA molecules for adaptor addition or linkage presents an effective strategy for their depletion. Successful circular RNA enrichment can be achieved by efficiently attaching 3′ and 5′ adaptors to the corresponding 3′ and 5′ ends of linear RNA molecules. The method further includes the sub-steps of treating the RNA sample with enzymes, ligases, or their combinations, as well as other enzymes commonly known in the art. Herein, enzymes can add or remove chemical moieties from 3′ and 5′ free ends of the linear RNA molecules. Herein, kinases or ligases or enzymes can add adaptor, linker or oligonucleotides efficiently to 3′ and 5′ ends of the linear RNA molecules for their depletion. Herein, the functional ends of 3′ or 5′ adaptors, linkers, or oligonucleotides can be adjusted or modified to ensure compatibility with the functional ends of linear RNA in the sample, allowing for their efficient attachment to linear RNA molecules.

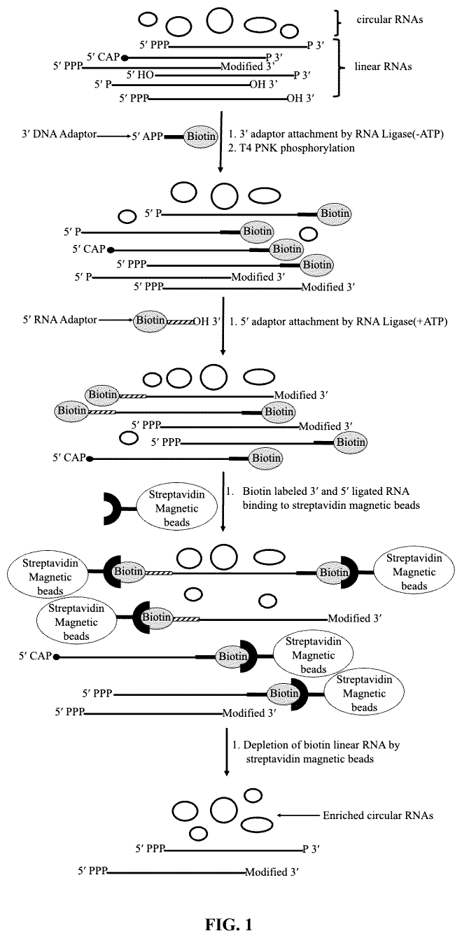

In one embodiment, the total RNA comprises all RNA molecules extracted from a biological sample, including both linear RNAs and circular RNAs. In the first step, total RNA, containing linear RNAs with a 3′-OH end, is ligated to the 5′ end of a 3′ adaptor having a adenylated 5′ end (5′-APP). In the next step 5′-monophosphate ends of linear RNA is ligated to the 3′-OH end of a 5′-adaptor. Both the 3′ and 5′ adaptors contain biotin affinity molecules, which become integrated into the linear RNA when attached to its ends. Streptavidin-coated paramagnetic beads are subsequently added to the sample, capturing the RNA-biotin-streptavidin complexes. A magnet is then applied, allowing easy removal of bead-captured linear RNA from the total RNA sample. The linear RNA-depleted sample contain enriched circular RNAs then purified using RNA purification kit. Enrichment method as shown in the FIG. 1 and is described in the Example 1.

In one embodiment depleted linear RNA transcripts were quantified using RT-qPCR. Linear RNA depletion was carried out as described in example 1. Several linear RNA such as abundant (GAPDH, ACTB, 12S mitochondrial, 18S-rRNA), Small Nuclear RNA (U1, U2, U5) and several other linear RNA transcripts were quantified by RT-qPCR as given in the example 1. The method demonstrated in this invention enables highly efficient depletion of linear RNAs. RT-qPCR details are described in the Example 1.

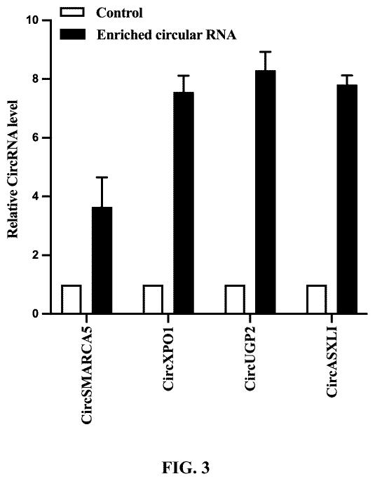

In one embodiment circular RNA enrichment was carried as demonstrated in example 1. Several enriched circular RNAs (CircSMARCA5, CircXPOL1, CircUGP2 and CircASOXL1) were quantified using RT-qPCR. The method as disclosed in the present invention provides the highly efficient enrichment of circular RNAs. Data is shown in the FIG. 3.

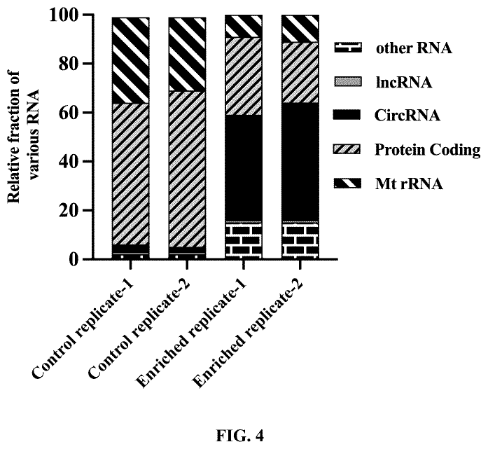

In one embodiment the classification of analyzed sequence reads from RNA-seq libraries, categorizing the sequencing reads into various RNA types, such as protein coding RNA, mitochondrial RNA (mt-RNA), long non-coding RNA (lncRNA), and other types of RNA. Distribution of sequencing reads from libraries prepared using control (total RNA) and enriched (with sequential 3′ and 5′ adaptor ligation) samples. The method according to the present invention demonstrate a highly efficient enrichment of circular RNAs accounting for ˜40-50% in all enriched libraries. Additionally, some of the protein coding reads are also associated with enriched circular RNAs. The compositions and methods disclosed in the present invention (Example 1) exhibited significantly higher percentages of circular RNA reads (FIG. 4) compared to control total RNA.

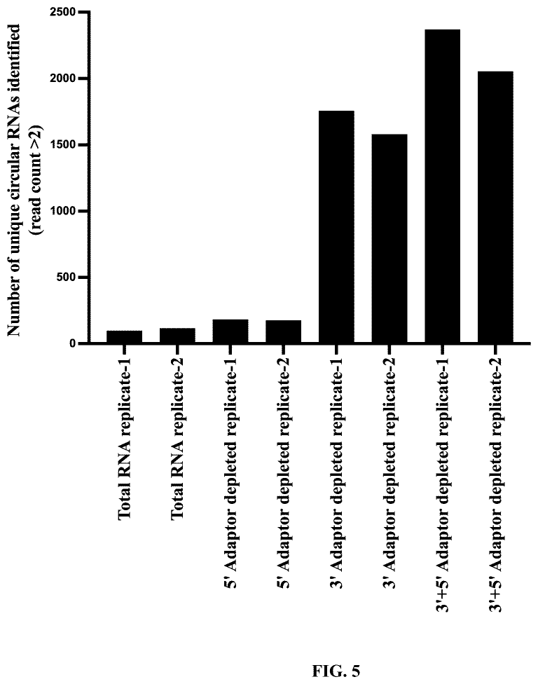

In one embodiment the number of unique circular RNAs enriched using various enrichment methods described in this invention. Circular RNAs with more than 2 reads were counted. A total of 4 μg of starting total RNA was used for all methods (Examples 1-3). As demonstrated in this invention, the sequential use of both 3′ and 5′ adaptors is more effective in depleting linear RNAs compared to the use of either 5′ or 3′ adaptors individually. 5′ Adaptor depletion method was the least effective in depleting linear RNAs (Example 3). Over 2000 unique circular RNAs were identified when both 3′ and 5′ adaptors were employed to deplete linear RNA (FIG. 5).

Further, disclosed herein use T4 PNK for generating 3′-hydroxyl ends of linear RNA molecules suitable for 3′ adaptor addition. The RNA sample contains linear RNA molecules with unsuitable 3′ ends, including but not limited to 3′-monophosphate, 3′-diphosphate, 3′-triphosphate, or 2′-3′ cyclic phosphate groups. In certain preferred embodiments, the RNA sample, containing various 3′ chemical moieties, is treated with enzymes to produce a 3′-hydroxyl group necessary for efficient 3′ adaptor addition or linking.

Further, disclosed herein use enzymes pyrophosphohydrolase (RppH) and T4 PNK for generating 5′-monophosphate ends of linear RNA molecules which are necessary for 5′ adaptor addition. For example, RppH is an enzyme that catalyzes the removal of the pyrophosphate or phosphate group from the 5′ end of triphosphorylated and diphosphorylated RNA, converting it into RNA with a 5′ monophosphate. Many linear RNA molecules in a sample contains undesired 5′ end that have 5′ triphosphate, 5′ diphosphate, 5′ hydroxyl or 5′ capped groups. Thus, in some preferred embodiment RNA sample consisting of various functional moieties at 5′ end; wherein treated with enzymes to generate 5′ monophosphate or 5′ phosphate.

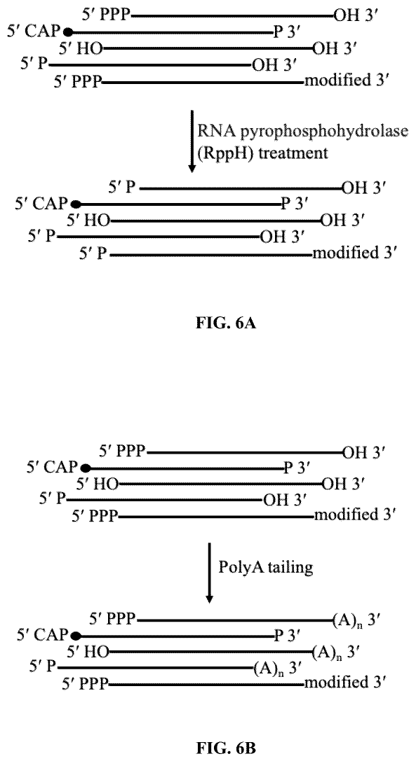

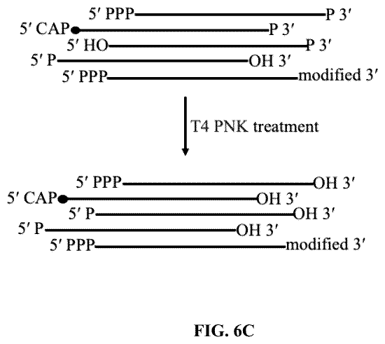

In one embodiment, the depletion of linear RNAs is demonstrated by modifying their 3′ and 5′ ends with specific enzymes to enhance ligation efficiency. By utilizing enzymatic treatments at both ends of the linear RNA molecules, the invention enables effective linear RNA depletion, thereby enhancing circular RNA enrichment. As demonstrated in the FIGS. 6A-6C, the total RNA was initially treated with enzymes, including RppH, Poly(A) polymerase, and T4 PNK, to modify the 3′ and 5′ ends of the linear RNA. As depicted in the FIG. 7, the depleted linear RNAs, including 12S mitochondrial RNA, 18S rRNA, GAPDH, SH38P5, and U5 (a small non-coding RNA), were selected as representative linear RNAs to assess the depletion efficiency of the methods and compositions demonstrated in the invention. Composition and methods demonstrated in the invention efficiently deplet abundant linear RNA molecules.

In certain aspects, disclosed herein is the use of T4 PNK to transfer a phosphate group from ATP to the 5′-hydroxyl end of RNA molecules, converting it to a 5′-monophosphate. This modification is crucial for subsequent processes such as ligation, as many ligases require a 5′-monophosphate to create a phosphodiester bond between two RNA molecules. T4 PNK also exhibits 3′ phosphatase activity, which removes phosphate groups from the 3′-phosphate ends of nucleic acids, leaving a 3′-hydroxyl group. This modification is essential for RNA molecules that need further ligation, as many enzymes involved in RNA 3′ end modifications or additions require a free 3′—OH group.

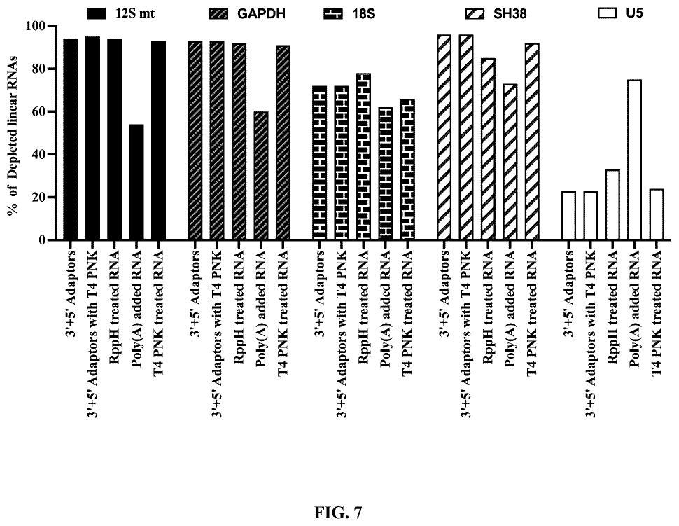

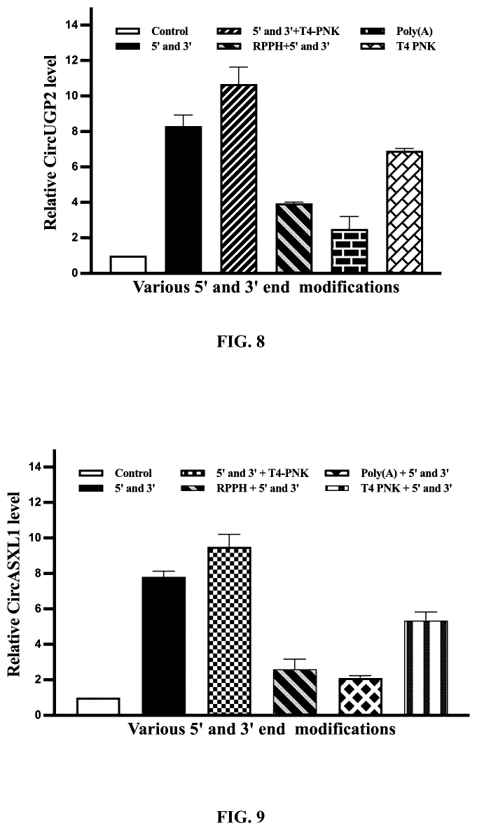

One embodiment demonstrates the enrichment of circular RNAs by utilizing methods that manipulate the 3′ and 5′ ends of various linear RNA molecules with specific enzymes to enhance circular RNA enrichment efficiency. As demonstrated in the FIG. 6, the total RNA was initially treated with enzymes, including RppH, Poly(A) polymerase, and T4 PNK, to modify the 3′ and 5′ ends of the linear RNA. The representative circular RNAs, circUGP2 and circASXOL1, are two of over 2,000 circular RNAs identified in the present invention. FIG. 8 and FIG. 9 results demonstrate that the composition and methods employed in the invention efficiently enrich circular RNAs by depleting linear RNAs.

In certain aspects, disclosed herein a method for depleting linear RNAs from a total RNA sample is provided. The method involves contacting the sample with T4 RNA ligase 2 truncated and 3′ adaptors that have a 5′-APP (adenyl pyrophosphoryl) end, enabling efficient addition or ligation to the 3′ end of linear RNA without the need for ATP. This approach is especially beneficial for preventing intermolecular linear RNA ligation, which can occur when ATP and T4 RNA ligase 1 are used in the reaction. The method may comprise contacting the sample with one or more 3′-adaptors to promote their addition to several unattached linear RNA molecules, whose addition was previously inhibited due to structural perturbation. The 3′ adaptors may possess variable or optimized functional 5′ ends, as well as variable nucleotide sequences, to enhance their efficient ligation to the 3′ ends of the target linear RNA.

In certain aspects, a method for depleting linear RNAs using 5′-adaptor addition from a total RNA sample is provided. The method involves contacting the sample with one or more 5′-adaptors to facilitate their efficient addition. The adaptors may have variable or optimized 3′ ends to facilitate their efficient addition to 5′-ends of linear RNA. The 5′ adaptors may possess variable or optimized functional 3′ ends, as well as variable nucleotide sequences, to facilitate their efficient ligation to the 5′ ends of linear RNA.

In one embodiment, the present invention the 5′ or 3′ adaptor addition or linking to linear RNAs can be enzymatic or chemical or their combinations.

In certain aspects, the removal of ribosomal RNA (rRNA) using alternative methods ensures that the sample provided for 5′ and 3′ ligation or addition is substantially free of rRNA, allowing for more efficient 5′ and 3′ addition to less abundant linear RNA molecules in the sample. The inclusion of an additional rRNA depletion step prior to linear RNA ligation and enrichment serves to enhance the enrichment of circular RNA.

In another embodiment, those with the knowledge in the art will know and understand other methods for 3′ and 5′ adaptor or linker or nucleotide or oligonucleotide addition to 3′ and 5′ ends of linear RNA molecules. All of which methods are within the scope of the present invention.

In one embodiment, the method utilizes 3′ and 5′ adaptors that are biotinylated, although the incorporation of other affinity molecules is also contemplated within the scope of the invention. In certain aspects, affinity molecules or labels within adaptors, linkers, nucleotides, or oligonucleotides may be utilized to deplete linear RNA attached to them. These affinity molecules or labels may include, but are not limited to, sulfhydryl-labeled nucleotides, aminoallyl-labeled nucleotides, and nucleotides containing allyl or azide functional groups. To deplete linear RNA, a capture agent may bind to a label that is covalently linked or present within adaptors, linkers, nucleotides, or oligonucleotides.

In one embodiment, the length of the 3′ adaptor is 50 nucleotides (nt) or less, preferably 30 nt or less, and more preferably 20 nt or less. The adaptor may have a biotin molecule attached at the 3′ end or at other positions along the adaptor sequence. The 5′ end of the adaptor may include APP (adenylated modification) group, or other chemical modifications to facilitate efficient ligation to the 3′ end of the RNA molecule. Furthermore, the 3′ end of the adaptor can include a dideoxycytidine (ddC) modification to prevent inter- and intra-molecular ligation or circularization, thereby improving the efficiency of 3′ ligation or addition reactions.

In one embodiment, the 3′ and 5′ adaptors, linkers, or oligonucleotides may include a group of molecules comprising 1-4, 3-10, 4-6, or combinations thereof to facilitate efficient 3′ and 5′ addition.

In certain embodiments, the methods, compositions, and kits described herein may be employed to detect and/or quantify the expression of circular RNAs prepared from cell extracts, whole cells, samples from in vitro transcription reactions, and samples from chemical synthesis. In certain embodiments, the disclosed methods, compositions, and kits can be utilized to detect and/or measure the expression levels of circular RNAs in biological samples. These methods are also applied to identify and validate circular RNAs biomarkers for disease detection and monitoring. Additionally, the methods can be employed to screen RNA samples from individuals or populations under various health conditions, ages, or other relevant circumstances to detect potential circular RNA biomarkers.

Total RNA may be isolated from a sample of interest utilizing methods well established in the prior art to obtain an initial composition comprising circular RNA. The term “sample” is used broadly to encompass various sources and compositions that contain circular RNA. The sample may be a biological sample, including, but not limited to, cell samples, human body fluids, human, animal, and plant tissues, environmental samples, and samples from viruses and micro-organisms.

In one embodiment, present invention provides a circRNA enrichment kit for depleting linear RNAs from total RNA containing sample. The kit includes:
(i) RNA ligation enzymes;
(ii) RNA ligation buffers;
(iii) 3′-Biotin-adaptors and 5′-Biotin adaptors;
(iv) Streptavidin magnetic beads;
(v) Biotin binding and washing buffers; and
(vi) Instruction to use the kits.

A method of enriching circular RNAs using the kits disclosed herein includes the following steps:
(a) isolation of total RNA which is free of DNA;
(b) treatment of total RNA with various end manipulating enzymes to make them suitable for their 3′- and 5′-adaptor addition;
(c) performing 3′ ligation reaction with 3′-adaptor in a suitable ligation condition;
(d) performing 5′ ligation reaction with 5′-adaptor in a suitable ligation condition;
(e) performing affinity depletion for ligated non-circular RNAs and obtaining enriched circular RNAs in the supernatant;
(f) isolating enriched circular RNA using RNA isolation kit; and
(g) preparing sequencing library and cDNA for circular RNA quantification and identification.

All technical and scientific names and terms used herein have the same meaning as commonly understood in the research field. The materials, methods and examples are illustrative purpose only and not intended to be limiting.

Unless explicitly indicated otherwise, the methods and compositions presented herein are not confined to the particular methodologies or reagents discussed but are intended as illustrative examples. Numerous aspects are described for clarification purposes. Process steps that involve standard and commonly known techniques to those skilled in the field are not elaborated on in detail.

Circular RNA isolation kit may include necessary materials and reagents for their enrichment or isolation from a sample. Kit also includes solution(s) and/or component(s) to provide the necessary reaction conditions. This generally will include a targeting agent, such as biotinylated adaptors or oligonucleotide or linker. The kit will also include instructions for using the kit components, as well as guidance on any additional reagents not provided in the kit. The instructions may include variations that can be implemented as needed.

The following examples are provided to illustrate various embodiments of the invention and are not intended to limit the invention in any way. These examples, along with the methods described herein, represent preferred embodiments and are exemplary, but should not be construed as limitations on the scope of the invention. Modifications and other uses that fall within the spirit of the invention, as defined by the claims, will be apparent to those skilled in the art.

### Example 1

### Reagents and Materials

(a) Nuclease-free 1.7-ml microcentrifuge tubes (Denville Scientific, catalog number: C2171)
(b) ThermoGrid™ rigid strip 0.2-ml PCR tubes (Denville Scientific, catalog number: C18064 (1000859)
(c) MicroAmp® Fast optical 96-well reaction plate (Thermo Fisher Scientific, Applied Biosystems™, catalog number: 4346906)
(d) Optical adhesive film (Thermo Fisher Scientific, Applied Biosystems™, catalog number: 4311971)
(e) Dulbecco's phosphate-buffered saline (DPBS) (Thermo Fisher Scientific, Gibco™, catalog number: 14040-133)
(f) Monarch® Total RNA Miniprep Kit (New England Biolabs, Ipswich, US-MA, catalog number: T2010S)
(g) Nuclease-free water (Thermo Fisher Scientific, Invitrogen™, catalog number: AM9930)
10(h) RiboLock RNase inhibitor (40 U/μl) (Thermo Fisher Scientific, Thermo Scientific™, catalog number: EO0381)
(i) dNTP mix (10 mM each) (Thermo Fisher Scientific, Thermo Scientific™, catalog number: R0193)
(j) SuperScript III First-Strand Synthesis System (Invitrogen, catalog number: 18091050)
(k) T4 RNA ligase 2, truncated KQ (New England Biolabs, catalog number: M0373L)
(l) T4 RNA ligase 1 (New England Biolabs, catalog number: M0204S)
(m) T4 Polynucleotide Kinase (New England Biolabs, catalog number: M0201S)
(n) Poly(A) Tailing Kit (Thermo Fisher Scientific, catalog number: AM1350)
(o) Murine RNase inhibitor (New England Biolabs, catalog number: M0314L)
(p) Monarch RNA Cleanup Kit (New England Biolabs, catalog number: T2030L)
(q) Qubit RNA High Sensitivity (HS) Assay Kit (Invitrogen, catalog number: Q32852,)
(r) NEBNext® Ultra™ II Directional RNA Library Prep Kit for Illumina® (New England BioLabs, catalog number: E7760S)
(s) Hydrophilic Streptavidin Magnetic Beads (New England BioLabs, catalog number: S1421S).

### Buffers and Reagents Used in the Experiment:

Ligation buffer: 50 mM Tris HCl pH 7.5; 1 mM DTT; 10 mM MgCl₂.
1× Streptavidin Magnetic Beads binding buffer: 10 mM Tris HCl pH 7.5; 0.5M NaCl and EDTA 1 mM.

### Hela Cell Culture

Hela cells were cultured in a 37° C., 5% CO₂ and 20% O₂ humidified incubator with Dulbecco's Modified Eagles Medium (DMEM, Gibco) containing high glucose (Thermo Fisher Scientific 25030081) supplemented with 10% Fetal Bovine Serum (FBS, Thermo Fisher), 1% penicillin/streptomycin (Penn/Strep, Thermo Fisher) and 1% L-glutamine.

### RNA Isolation

Total cell RNA was isolated using Monarch Total RNA Miniprep Kit (New England BioLabs) following the manufacturer's instructions, including the DNase I treatment step. RNA concentration was determined by a NanoDrop 2000 spectrophotometer (Thermo Fisher Scientific) and Qubit RNA High Sensitivity assay.

Enriching Circular RNAs from Total RNA Sample Using 3′-Adaptor and 5′-Adaptor

A 20 μL reaction was set up containing 4 μg of total RNA, 40 U of Murine RNase inhibitor (NEB #M0314L), 50 pmol of 3′-adaptor (The 3′ adaptor comprises the sequence SEQ ID NO: 1, which includes the RNA base with a 5′-adenylated form (App) modification and a 3′ biotin (Bio) modification, having the following DNA bases sequence: CTGTAGGCACCATCAAT.), 10 U of T4 RNA ligase-2 truncated, with 30% PEG 8000 in RNA ligation buffer (1×50 mM Tris-HCl, 2 mM MgCl₂, 1 mM DTT). The reaction was incubated at 37° C. for 30 minutes, followed by 4 hours at 24° C. Subsequently, 50 pmol of 5′-adaptor (The 5′ adaptor comprises the sequence SEQ ID NO: 2, which includes all RNA bases with a 5′ biotin (Bio) modification and the following sequence: rGrUrUrCrArGrArGrUrUrCrUrArCrArGrUrCrCrGrArCrGrArUrC.), 10 U of T4 RNA ligase-1, and 1 mM ATP were added to the same reaction, which was then incubated for 2 hours at 24° C. and 2 hours at 16° C.

The Hydrophilic Streptavidin Magnetic Beads (New England BioLabs, #S1421S) were gently vortexed to ensure uniform resuspension, and a 100 μL aliquot of the beads was transferred into a clean microcentrifuge tube. The tube was placed on a magnetic separator rack to allow the beads to settle along the side, and the supernatant was carefully removed without disturbing the beads. For washing, 300 μL of binding buffer (20 mM Tris, pH 7.5, 500 mM NaCl, 1 mM EDTA) was added, and the beads were gently resuspended by pipetting. The tube was returned to the magnetic rack to collect the beads, and the supernatant was carefully removed. This washing step was repeated two more times, for a total of three washes. The beads were then resuspended in 100 μL of binding buffer. A 20 μL reaction mixture was added to the beads, followed by a 20-minute incubation at room temperature, with gentle pipetting every 5 minutes to ensure optimal binding. After incubation, the tube was placed on the magnetic rack to collect the beads, and the supernatant, containing the unbound molecules, was carefully removed for subsequent RNA isolation. The enriched RNA was purified using the Monarch RNA Cleanup Kit (NEB #T2030L), and RNA was eluted in 8 μL of RNase-free water. The RNA concentration was then quantified using a NanoDrop spectrophotometer and Qubit RNA High Sensitivity assay.

cDNA for RT-qPCR

For all cDNA synthesis reactions, 100 ng of RNA was used to standardize the amount across samples. The isolated RNA was reverse transcribed to generate first-strand cDNA using the SuperScript™ III First-Strand Synthesis System (Invitrogen, 18091050) according to the manufacturer's protocol. The 20 μL reaction mixture contained 100 ng of RNA, 2 μL of 10× buffer, 1 μL of 10 mM dNTP mixture, 2 μL of 100 mM DTT, 4 μL of 10 mM MgCl₂, 1 μL of RNaseOUT (40 U/μL), 1 μL of random primer, 1 μL of SuperScript III RT (200 U/μL), and 6.5 μL of DEPC-treated water. The thermal cycling conditions were set to 25° C. for 10 minutes, 50° C. for 50 minutes, followed by reverse transcriptase inactivation at 85° C. for 20 minutes. Subsequently, 1 μL of RNase H was added, and the reaction was incubated at 37° C. for 20 minutes. The final reaction volume was adjusted to 60 μL by adding 39 μL of water.

### RT-qPCR

Following reverse transcription, quantitative PCR (qPCR) was performed using the Luna Universal qPCR Master Mix (NEB #M3003) on the QuantStudio 5 Real-Time PCR System. The reaction mixture consisted of 1 μL of cDNA template, 0.5 μL of each primer (10 uM), 10 μL of 2× Luna Universal qPCR Master Mix, and 8 μL of distilled water, with two technical replicates for each cDNA sample. The qPCR program included an initial denaturation at 95° C. for 60 seconds, followed by 40 cycles of denaturation at 95° C. for 10 seconds and annealing/extension at 60° C. for seconds. The percentage of linear RNA remaining was determined according to the published method [22]. The relative expression levels of each gene were calculated using the 2^{-ΔΔCt} method. The ΔCt value was obtained by subtracting the Ct value of control from the Ct value of the target genes. The reaction's validation was assessed through the melting curve (single peak). Each isolation method was analyzed in triplicates.

GAPDH Forward primer (linear)
(SEQ ID NO: 3)
GGTGGTCTCCTCTGACTTCAACA
GAPDH Reverse primer (linear)
(SEQ ID NO: 4)
GTTGCTGTAGCCAAATTCGTTGT
ACTB Forward primer (linear)
(SEQ ID NO: 5)
CATGTACGTTGCTATCCAGGC
ACTB Reverse primer (linear)
(SEQ ID NO: 6)
CTCCTTAATGTCACGCACGAT
18S Forward primer (linear)
(SEQ ID NO: 7)
CGAACGTCTGCCCTATCAACTT
18S Reverse primer (linear)
(SEQ ID NO: 8)
ACCCGTGGTCACCATGGTA
12S-mt Forward primer (linear)
(SEQ ID NO: 9)
TAGATACCCCACTATGCTTAGC
12S-mt Reverse primer (linear)
(SEQ ID NO: 10)
CGATTACAGAACAGGCTCC
U1 Forward primer (linear)
(SEQ ID NO: 11)
CCACAAATTATGCAGTCAAGTTTCCCA
U1 Reverse primer (linear)
(SEQ ID NO: 12)
CCATGATCACGAAGGTGGTTTTCC
U2 Forward primer (linear)
(SEQ ID NO: 13)
CTGATACGTCCTCTATCCGAGGACA
U2 Reverse primer (linear)
(SEQ ID NO: 14)
GGTCGATGCGTGGAGTGGAC
U5 Forward primer (linear)
(SEQ ID NO: 15)
CTTCAGATCGCATAAATCTTTCGCCTT
U5 Reverse primer (linear)
(SEQ ID NO: 16)
CAAAAAATTGGGTTAAGACTCAGAGTTGTTCC
YRNA-3 Forward primer (linear)
(SEQ ID NO: 17)
GTGCAGTGGTGTTTACAACTAATTGATCA
YRNA-3 Reverse primer (linear)
(SEQ ID NO: 18)
GTCAAGTGAAGCAGTGGGAGTGG
RPPH1 Forward primer (linear)
(SEQ ID NO: 19)
CTAACAGGGCTCTCCCTGAGC
RPPH1 Reverse primer (linear)
(SEQ ID NO: 20)
GTTCCAAGCTCCGGCAAAGG
SH3BP5 Forward primer (linear)
(SEQ ID NO: 21)
TGAAGCAGCTCTCCCTACAGTG
SH3BP5 Reverse primer (linear)
(SEQ ID NO: 22)
TGGCACAATGTTCTCCAGTTCC
tRNA-Gln-TTG Forward primer (linear)
(SEQ ID NO: 23)
GGACATGGTGTGATAGGTAGCAT
RNA-Gln-TTG Reverse primer (linear)
(SEQ ID NO: 24)
AGGAATTGAACCTACCCCTGA
tRNA-Leu-TAA Forward primer (linear)
(SEQ ID NO: 25)
ATGGCAGAGCCTGGTAATTG
tRNA-Leu-TAA Reverse primer (linear)
(SEQ ID NO: 26)
AAGAAGAGGAGTTGAACCTCTGA
PPP2R4 Forward primer (linear)
(SEQ ID NO: 27)
CCCGTTTGATGAGAGGCTGTTTAC
PPP2R4 Reverse primer (linear)
(SEQ ID NO: 28)
AGTCTAGCACACGGGAACTCAG
FSTL3 Forward primer (linear)
(SEQ ID NO: 29)
AGGGAAGGCAGTGGACAAGG
FSTL3 Reverse primer (linear)
(SEQ ID NO: 30)
TCCAGACTGATGTCACCCGG
GANAB Forward primer (linear)
(SEQ ID NO: 31)
TTATCACCACCCGCTCAATCCAG
GANAB Reverse primer (linear)
(SEQ ID NO: 32)
AGACTCGCCAAGAGTTCCTGC
HIST2H2AB Forward primer (linear)
(SEQ ID NO: 33)
GTTCTTGCCAGGCTTGTGACTC
HIST2H2AB Reverse primer (linear)
(SEQ ID NO: 34)
ACTAGCCGTGAGGAATGACGAAG
circSMARCA5 Forward primer (circular)
(SEQ ID NO: 35)
TCCAAGATGGGCGAAAGTTCACT
circSMARCA5 Reverse primer (circular)
(SEQ ID NO: 36)
GTTCAGATTCTGATCCACAAGCCTC
circXPOL1 Forward primer (circular)
(SEQ ID NO: 37)
GGCTGAAATCAAGCAGCTGACG
circXPOL1 Reverse primer (circular)
(SEQ ID NO: 38)
CTTCCAAGGAACCAGTGCGAA
circUGP2 Forward primer (circular)
(SEQ ID NO: 39)
CTTCTGTGGATTGGGGAAAA
circUGP2 Reverse primer (circular)
(SEQ ID NO: 40)
TGTGATGATGCTGTGGTGAG
circASXL1 Forward primer (circular)
(SEQ ID NO: 41)
TATAAACTGCCTGGCCGAAT
circASXL1 Reverse primer (circular)
(SEQ ID NO: 42)
TCCCACTGCTCATTTCCTTT

### Example 2

Enriching Circular RNAs from Total RNA Sample Using 3′-Adaptor

A 20 μL reaction was set up containing 4 μg of total RNA, 40 U of Murine RNase inhibitor (NEB #M0314L), 50 pmol of 3′-adaptor, 10 U of T4 RNA ligase-2 truncated, with 30% PEG 8000 in RNA ligation buffer (1×50 mM Tris-HCl, 2 mM MgCl₂, 1 mM DTT). The reaction was incubated at 37° C. for 30 minutes, followed by 4 hours at 24° C. Subsequently, 10 U of T4 RNA ligase-1, and 1 mM ATP were added to the same reaction, which was then incubated for 2 hours at 24° C. and 2 hours at 16° C. Circular RNAs were enriched by depleting linear RNAs using streptavidin beads by following the protocol described in the Example 1.

### Example 3

Enriching Circular RNAs from Total RNA Sample Using 5′-Adaptor

A 20 μL reaction was set up containing 4 μg of total RNA, 40 U of Murine RNase inhibitor (NEB #M0314L), 10 U of T4 RNA ligase-2 truncated, with 30% PEG 8000 in RNA ligation buffer (1×50 mM Tris-HCl, 2 mM MgCl₂, 1 mM DTT). The reaction was incubated at 37° C. for 30 minutes, followed by 4 hours at 24° C. Subsequently, 50 pmol of 5′-adaptor, 10 U of T4 RNA ligase-1, and 1 mM ATP were added to the same reaction, which was then incubated for 2 hours at 24° C. and 2 hours at 16° C. Circular RNAs were enriched by depleting linear RNAs using streptavidin beads by following the protocol described in the Example 1.

### Example 4

Enriching Circular RNAs from Total RNA Sample Using 3′-Adaptor and 5′-Adaptor in the Presence of T4 Polynucleotide Kinase (T4 PNK)

A 20 μL reaction was set up containing 4 μg of total RNA, 40 U of Murine RNase inhibitor (NEB #M0314L), 10U T4 PNK, 50 pmol of 3′-adaptor, 10 U of T4 RNA ligase-2 truncated, with 30% PEG 8000 in RNA ligation buffer (1×50 mM Tris-HCl, 2 mM MgCl₂, 1 mM DTT). The reaction was incubated at 37° C. for 30 minutes, followed by 4 hours at 24° C. Subsequently, 50 pmol of 5′-adaptor, 10 U of T4 RNA ligase-1, and 1 mM ATP were added to the same reaction, which was then incubated for 2 hours at 24° C. and 2 hours at 16° C. Circular RNAs were enriched by depleting linear RNAs using streptavidin beads by following the protocol described in the Example 1.

### Example 5

### Generation of 5′ Monophosphate RNA by RppH Enzyme

For 5′ monophosphate generation of linear RNA 4 ug total RNA was incubated with 20U RppH enzyme for 30 min at 37° C. in the 40 ul reaction in NEB buffer 2. The RNA was purified using the Monarch RNA Cleanup Kit (NEB #T2030L), and RNA was eluted in 6 μL of RNase-free water. RPPH treated total RNA was used to enriched circular RNAs by following the protocol described in the Example 1.

### Example 6

### Poly(A) Tailing

A total of 4 μg of RNA was subjected to poly(A) tailing in a 20 μL reaction using the Poly(A) Tailing Kit (Thermo Fisher Scientific, #AM1350) according to the manufacturer's instructions. The reaction mixture contained 4 μL of 5× E-PAP buffer, 4 μL of 25 mM MnCl₂, 4 μL of 10 mM ATP solution, 1 μL of RiboLock RNase inhibitor (Thermo Fisher Scientific, #N8080119), and 1.0 μL of E-PAP (2 U/μL), and was incubated for 30 minutes at 37° C. Following incubation, the RNA was purified using the Monarch RNA Cleanup Kit (NEB #T2030L). Poly(A) RNA was eluted in 6 μL of RNase-free water and quantified by nanodrop spectrophotometer. Poly(A) tailed total RNA was used to enriched circular RNAs by following the protocol described in the Example 1.

### Example 7

### Generation of 5′ Monophosphate and 3′-OH Ends RNA by T4 PNK

A total of 10 μg of RNA was incubated with 5 μL of T4 PNK enzyme and 5 μL of RiboLock RNase inhibitor for 30 minutes at 37° C. in a 50 μL reaction using T4 PNK buffer. Afterward, 1 mM ATP was added to the reaction, and incubation continued for an additional 30 minutes at 37° C. The RNA was then purified using the Monarch RNA Cleanup Kit (NEB #T2030L) and eluted in 6 μL of RNase-free water. T4 PNK treated total RNA was used to enriched circular RNAs by following the protocol described in the Example 1.

### Library Preparation and Sequencing

The cDNA libraries were prepared by NEBNext® Ultra™ II Directional RNA Library Prep Kit for Illumina® (NEB, USA, #E7760S) following manufacturer's recommendations. The library was sequenced by Element Aviti platform sequencer using a 2×150 bp paired-end pattern (PE150).

### CircRNA Prediction and Transcriptome Analysis by Bioinformatic Analysis

To detect circular RNAs with CIRI2[23], RNA-seq reads were initially aligned to the hg38 genome using bwa, and the resulting alignments were analyzed in CIRI2 with its default settings. Circular RNA candidates identified by CIRI2 were required to have at least two junction reads in one library to be considered for analysis.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### Patents

CN111607588A
CN110283895A
WO2018149091A1

### REFERENCES

1. Salzman J, Chen R E, Olsen M N, Wang P L, Brown P O. Cell-type specific features of circular RNA expression. PLOS Genet. 2013; 9(9): e1003777.
2. Jeck W R, Sharpless N E. Detecting and characterizing circular RNAs. Nat Biotechnol. 2014 May; 32(5): 453-61.
3. Liu C X, Chen L L. Circular RNAs: Characterization, cellular roles, and applications. Cell. 2022, Jun. 9; 185(12): 2016-2034.
4. Salzman J. Circular RNA Expression: Its Potential Regulation and Function. Trends Genet. 2016 May; 32(5): 309-316.
5. Chen L L. The expanding regulatory mechanisms and cellular functions of circular RNAs. Nat Rev Mol Cell Biol. 2020 August; 21(8): 475-490.
6. Hansen T B, Jensen T I, Clausen B H, Bramsen J B, Finsen B, Damgaard C K, Kjems J. Natural RNA circles function as efficient microRNA sponges. Nature. 2013 Mar. 21; 495(7441): 384-8.
7. Zhang Y, Wang Y. Circular RNAs in Hepatocellular Carcinoma: Emerging Functions to Clinical Significances. Front Oncol. 2021 May 14; 11:667428.
8. Mehta S L, Dempsey R J, Vemuganti R. Role of circular RNAs in brain development and CNS diseases. Prog Neurobiol. 2020 March; 186:101746.
9. Wilusz J E. Circle the Wagons: Circular RNAs Control Innate Immunity. Cell. 2019 May 2; 177(4): 797-799.
10. Liu C X, Li X, Nan F, Jiang S, Gao X, Guo S K, Xue W, Cui Y, Dong K, Ding H, Qu B, Zhou Z, Shen N, Yang L, Chen L L. Structure and Degradation of Circular RNAs Regulate PKR Activation in Innate Immunity. Cell. 2019 May 2; 177(4): 865-880.e21.
11. Ligresti G, Pham T X, Sanders Y Y. Circular RNA Methylation: A New Twist in Lung Fibrosis. Am J Respir Cell Mol Biol. 2022 May; 66(5): 471-472.
12. Awan F M, Yang B B, Naz A, Hanif A, Ikram A, Obaid A, Malik A, Janjua H A, Ali A, Sharif S. The emerging role and significance of circular RNAs in viral infections and antiviral immune responses: possible implication as theranostic agents. RNA Biol. 2021 January; 18(1): 1-15.
13. Chen Y G, Chen R, Ahmad S, Verma R, Kasturi S P, Amaya L, Broughton J P, Kim J, Cadena C, Pulendran B, Hur S, Chang H Y. N6-Methyladenosine Modification Controls Circular RNA Immunity. Mol Cell. 2019 Oct. 3; 76(1): 96-109.e9.
14. Zhang Z, Yang T, Xiao J. Circular RNAs: Promising Biomarkers for Human Diseases. EBioMedicine. 2018 August; 34:267-274.
15. Szabo L, Salzman J. Detecting circular RNAs: bioinformatic and experimental challenges. Nat Rev Genet. 2016 Oct. 14; 17(11): 679-692.
16. Panda A C, De S, Grammatikakis I, Munk R, Yang X, Piao Y, Dudekula D B, Abdelmohsen K, Gorospe M. High-purity circular RNA isolation method (RPAD) reveals vast collection of intronic circRNAs. Nucleic Acids Res. 2017 Jul. 7; 45(12): e116.
17. Xiao M S, Wilusz J E. An improved method for circular RNA purification using RNase R that efficiently removes linear RNAs containing G-quadruplexes or structured 3′ ends. Nucleic Acids Res. 2019 Sep. 19; 47(16): 8755-8769.
18. Szabo L, Salzman J. Detecting circular RNAs: bioinformatic and experimental challenges. Nat Rev Genet. 2016 Oct. 14; 17(11): 679-692.
19. Westholm J O, Miura P, Olson S, Shenker S, Joseph B, Sanfilippo P, Celniker S E, Graveley B R, Lai E C. Genome-wide analysis of drosophila circular RNAs reveals their structural and sequence properties and age-dependent neural accumulation. Cell Rep. 2014 Dec. 11; 9(5): 1966-1980.
20. Zhang X O, Wang H B, Zhang Y, Lu X, Chen L L, Yang L. Complementary sequence-mediated exon circularization. Cell. 2014 Sep. 25; 159(1): 134-147.
21. Zhang Y, Yang L, Chen L L. Characterization of Circular RNAs. Methods Mol Biol. 2021; 2372:179-192.
22. Das A, Das D, Panda A C. Validation of Circular RNAs by PCR. Methods Mol Biol. 2022; 2392:103-114.
23. Gao Y. Zhang J, Zhao F. Circular RNA identification based on multiple seed matching. Brief Bioinform. 2018 Sep. 28; 19(5): 803-810.

1. A method of enriching circular ribonucleic acid (circRNA) molecules in a total RNA sample, the method comprising:
(a) obtaining the total RNA sample comprising the circRNA and non-circRNA molecules;
(b) contacting the sample with one or more adaptors comprising an affinity molecule under conditions to produce non-circRNA molecules ligated with the affinity molecule at one or more of 3′-end and 5′-end of the non-circRNA molecules;
(c) binding the ligated non-circRNA molecules to a binding matrix comprising the affinity molecule binding substance to capture and immobilize the ligated non-circRNA molecules from the total RNA sample;
(d) depleting the immobilized non-circRNA molecules by removing the binding matrix from the sample and thereby enriching the circRNA molecules in said total RNA sample.

2. The method of claim 1, wherein the method of enriching circRNA molecules in the total RNA sample further comprises treating the sample with a reagent mixture such that functionally reactive groups are generated at one or more of 3′-end and 5′-end of the non-circRNA molecules to ligate said adaptors comprising the affinity molecule.

3. The method claim 1, wherein said affinity molecule is biotin.

4. The method claim 1, wherein said binding matrix comprising the affinity molecule binding substance is streptavidin magnetic beads.

5. The method of claim 2, wherein the reagent mixture comprises T4 polynucleotide kinase, pyrophosphohydrolase, or poly(A) polymerase.

6. The method of claim 2, wherein said one or more functionally reactive groups of the non-circRNA molecules is selected from the group consisting of 3′-hydroxyl, 5′-hydroxyl, 3′-phosphate, and 5′-phosphate.

7. The method of claim 2, wherein the total RNA sample after treatment with the reagent mixture is optionally purified prior to ligating with said adaptors comprising the affinity molecule.

8. The method of claim 1, wherein said total RNA sample is isolated from a biological sample.

9. A method of enriching circular ribonucleic acid (circRNA) molecules in a total RNA sample, the method comprising:
(a) obtaining the total RNA sample comprising the circRNA and non-circRNA molecules;
(b) contacting the sample with one or more adaptors comprising a biotin label under conditions to produce non-circRNA molecules ligated with the biotin label at one or more of 3′-end and 5′-end of the non-circRNA molecules;
(c) binding the ligated non-circRNA molecules to streptavidin-coated magnetic beads to capture and immobilize the ligated non-circular RNA molecules via the biotin label;
(d) depleting the immobilized non-circRNA molecules by separating the magnetic beads from the sample and thereby enriching the circRNA molecules in said total RNA sample.

10. The method of claim 9, wherein the method of enriching circRNA molecules in the total RNA sample further comprises treating the sample with a reagent mixture such that functionally reactive groups are generated at one or more of 3′-end and 5′-end of the non-circRNA molecules to ligate said adaptors comprising the biotin label.

11. The method of claim 10, wherein the total RNA sample after treatment with the reagent mixture is optionally purified prior to ligating with said adaptors comprising the biotin label.

12. The method of claim 10, wherein said functionally reactive groups of the non-circRNA molecules is selected from the group consisting of 3′-hydroxyl, 5′-hydroxyl, 3′-phosphate, and 5′-phosphate.

13. The method of claim 10, wherein said reagent mixture comprises T4 polynucleotide kinase, pyrophosphohydrolase, or poly(A) polymerase.

14. The method of claim 10, wherein said total RNA sample is isolated from a biological sample.

15. A method of enriching circular ribonucleic acid (circRNA) molecules in a total RNA sample, the method comprising:
(a) obtaining the total RNA sample comprising the circRNA and non-circRNA molecules;
(b) contacting the sample with a reagent mixture such that one or more functionally reactive groups are generated at one or more of 3′-end and 5′-end of the non-circRNA molecules, wherein said one or more functionally reactive groups are selected from the group consisting of 3′-hydroxyl, 5′-hydroxyl, 3′-phosphate, and 5′-phosphate;
(c) contacting the sample with one or more adaptors comprising a biotin label under conditions to produce non-circRNA molecules ligated with the biotin label at one more of 3′-end and 5′-end of the non-circRNA molecules;
(d) binding the ligated non-circRNA molecules to streptavidin-coated magnetic beads to capture and immobilize the ligated non-circular RNA molecules via the biotin label;
(e) depleting the immobilized non-circRNA molecules by separating the magnetic beads from the sample and thereby enriching the circRNA in said total RNA sample.

16. The method of claim 15, wherein said reagent mixture comprises T4 polynucleotide kinase, pyrophosphohydrolase, or poly(A) polymerase.

17. The method of claim 15, wherein the one or more functionally reactive groups is 3′-hydroxyl.

18. The method of claim 15, wherein the one or more functionally reactive groups is 5′-monophosphate.

19. The method of claim 15, wherein the total RNA sample after treatment with the reagent mixture is optionally purified prior to ligating with said adaptors comprising the biotin label.

20. The method of claim 15, wherein said total RNA sample is isolated from a biological sample.


SEQUENCE LISTING (42 sequences)

SEQ ID NO: 1 - DNA, 17 bp, synthetic construct
  source 1..17: mol_type = other DNA
ctgtaggcaccatcaat

SEQ ID NO: 2 - RNA, 26 nt, synthetic construct
  source 1..26: mol_type = other RNA
gttcagagttctacagtccgacgatc

SEQ ID NO: 3 - DNA, 23 bp, synthetic construct
  source 1..23: mol_type = other DNA
ggtggtctcctctgacttcaaca

SEQ ID NO: 4 - DNA, 23 bp, synthetic construct
  source 1..23: mol_type = other DNA
gttgctgtagccaaattcgttgt

SEQ ID NO: 5 - DNA, 21 bp, synthetic construct
  source 1..21: mol_type = other DNA
catgtacgttgctatccaggc

SEQ ID NO: 6 - DNA, 21 bp, synthetic construct
  source 1..21: mol_type = other DNA
ctccttaatgtcacgcacgat

SEQ ID NO: 7 - DNA, 22 bp, synthetic construct
  source 1..22: mol_type = other DNA
cgaacgtctgccctatcaactt

SEQ ID NO: 8 - DNA, 19 bp, synthetic construct
  source 1..19: mol_type = other DNA
acccgtggtcaccatggta

SEQ ID NO: 9 - DNA, 22 bp, synthetic construct
  source 1..22: mol_type = other DNA
tagataccccactatgcttagc

SEQ ID NO: 10 - DNA, 19 bp, synthetic construct
  source 1..19: mol_type = other DNA
cgattacagaacaggctcc

SEQ ID NO: 11 - DNA, 27 bp, synthetic construct
  source 1..27: mol_type = other DNA
ccacaaattatgcagtcaagtttccca

SEQ ID NO: 12 - DNA, 24 bp, synthetic construct
  source 1..24: mol_type = other DNA
ccatgatcacgaaggtggttttcc

SEQ ID NO: 13 - DNA, 25 bp, synthetic construct
  source 1..25: mol_type = other DNA
ctgatacgtcctctatccgaggaca

SEQ ID NO: 14 - DNA, 20 bp, synthetic construct
  source 1..20: mol_type = other DNA
ggtcgatgcgtggagtggac

SEQ ID NO: 15 - DNA, 27 bp, synthetic construct
  source 1..27: mol_type = other DNA
cttcagatcgcataaatctttcgcctt

SEQ ID NO: 16 - DNA, 32 bp, synthetic construct
  source 1..32: mol_type = other DNA
caaaaaattgggttaagactcagagttgttcc

SEQ ID NO: 17 - DNA, 29 bp, synthetic construct
  source 1..29: mol_type = other DNA
gtgcagtggtgtttacaactaattgatca

SEQ ID NO: 18 - DNA, 23 bp, synthetic construct
  source 1..23: mol_type = other DNA
gtcaagtgaagcagtgggagtgg

SEQ ID NO: 19 - DNA, 21 bp, synthetic construct
  source 1..21: mol_type = other DNA
ctaacagggctctccctgagc

SEQ ID NO: 20 - DNA, 20 bp, synthetic construct
  source 1..20: mol_type = other DNA
gttccaagctccggcaaagg

SEQ ID NO: 21 - RNA, 22 nt, synthetic construct
  source 1..22: mol_type = other RNA
tgaagcagctctccctacagtg

SEQ ID NO: 22 - DNA, 22 bp, synthetic construct
  source 1..22: mol_type = other DNA
tggcacaatgttctccagttcc

SEQ ID NO: 23 - DNA, 23 bp, synthetic construct
  source 1..23: mol_type = other DNA
ggacatggtgtgataggtagcat

SEQ ID NO: 24 - DNA, 21 bp, synthetic construct
  source 1..21: mol_type = other DNA
aggaattgaacctacccctga

SEQ ID NO: 25 - DNA, 20 bp, synthetic construct
  source 1..20: mol_type = other DNA
atggcagagcctggtaattg

SEQ ID NO: 26 - DNA, 23 bp, synthetic construct
  source 1..23: mol_type = other DNA
aagaagaggagttgaacctctga

SEQ ID NO: 27 - DNA, 24 bp, synthetic construct
  source 1..24: mol_type = other DNA
cccgtttgatgagaggctgtttac

SEQ ID NO: 28 - DNA, 22 bp, synthetic construct
  source 1..22: mol_type = other DNA
agtctagcacacgggaactcag

SEQ ID NO: 29 - DNA, 20 bp, synthetic construct
  source 1..20: mol_type = other DNA
tccagactgatgtcacccgg

SEQ ID NO: 30 - DNA, 20 bp, synthetic construct
  source 1..20: mol_type = other DNA
agggaaggcagtggacaagg

SEQ ID NO: 31 - DNA, 23 bp, synthetic construct
  source 1..23: mol_type = other DNA
ttatcaccacccgctcaatccag

SEQ ID NO: 32 - DNA, 21 bp, synthetic construct
  source 1..21: mol_type = other DNA
agactcgccaagagttcctgc

SEQ ID NO: 33 - DNA, 22 bp, synthetic construct
  source 1..22: mol_type = other DNA
gttcttgccaggcttgtgactc

SEQ ID NO: 34 - DNA, 23 bp, synthetic construct
  source 1..23: mol_type = other DNA
actagccgtgaggaatgacgaag

SEQ ID NO: 35 - DNA, 23 bp, synthetic construct
  source 1..23: mol_type = other DNA
tccaagatgggcgaaagttcact

SEQ ID NO: 36 - DNA, 25 bp, synthetic construct
  source 1..25: mol_type = other DNA
gttcagattctgatccacaagcctc

SEQ ID NO: 37 - DNA, 22 bp, synthetic construct
  source 1..22: mol_type = other DNA
ggctgaaatcaagcagctgacg

SEQ ID NO: 38 - DNA, 21 bp, synthetic construct
  source 1..21: mol_type = other DNA
cttccaaggaaccagtgcgaa

SEQ ID NO: 39 - DNA, 20 bp, synthetic construct
  source 1..20: mol_type = other DNA
cttctgtggattggggaaaa

SEQ ID NO: 40 - DNA, 20 bp, synthetic construct
  source 1..20: mol_type = other DNA
tgtgatgatgctgtggtgag

SEQ ID NO: 41 - DNA, 20 bp, synthetic construct
  source 1..20: mol_type = other DNA
tataaactgcctggccgaat

SEQ ID NO: 42 - DNA, 20 bp, synthetic construct
  source 1..20: mol_type = other DNA
tcccactgctcatttccttt